(19) United States
(12) Patent Application Publication
(10) Pub. No.: US 2003/0204315 A1
(43) Pub. Date: Oct. 30, 2003

(54) System and method for carbohydrate sequence presentation, comparison and analysis

(76) Inventors: Avinoam Dukler, Modi'ln (IL); Nir Dotan, Shoham (IL)

Correspondence Address: G.E. EHRLICH (1995) LTD., c/o ANTHONY CASTORINA, SUITE 207, 2001 JEFFERSON DAVIS HIGHWAY, ARLINGTON, VA, 22202, US

(21) Appl. No.: 10/419,729

(22) Filed: Apr. 22, 2003

Related U.S. Application Data
(63) Continuation of application No. 09/573,548, filed on May 19, 2000.

(51) Int. Cl. G06F 19/00; G01N 33/48; G01N 33/50; G01R 23/16

(52) U.S. Cl. 702/19; 702/20

(57) ABSTRACT

A method for storing, retrieving, comparing and analyzing complex carbohydrates, by representing complex carbohydrates with a simple linear code, which is preferably also able to represent branches within the carbohydrate structure. The method of the present invention for converting the carbohydrate structure to such a linear code includes the steps of parsing each component of the structure; separately demarcating each branch within the structure; and then converting each component to a symbolic representation which may optionally be alphabetic, numeric, or a combination thereof.

### RELATED APPLICATION

This application is a continuation of U.S. patent application No. Ser. 09/573,548 filed May 19, 2000.

### FIELD AND BACKGROUND OF THE INVENTION

The present invention is related to a system and method for the presentation, analysis and comparison of carbohydrates, and in particular, to such a system and method in which complex carbohydrates/oligosaccharides are compared according to both sequence and structure, such that the carbohydrates are first converted to a linear representation of the sequence and structure thereof, before the comparison and/or analysis of the carbohydrates is performed.

Informatics

Informatics is basically information management as it relates to scientific research. The software tools, and related databases, which are provided through informatics enables the vast quantities of information to be managed, analyzed and maintained. Based on database and statistical techniques, informatics tools permit scientists to store, query, access, share, and use all of the data at their disposal. Without tools to handle, store, retrieve and analyze these data, data may be lost, duplicated, or simply never utilized.

With regard to the life sciences, informatics may be split into two disciplines: bioinformatics and cheminformatics. Bioinformatics is concerned with the management, organization, and use of the data that describe biological material, mainly proteins and DNA. Cheminformatics is concerned with the management, organization, and use of the data that describe chemical compounds, with regard to their structure and properties (Knapman, 1999).

Bioinformatics has emerged as a new branch of biology, following the advances made in experimental technologies of molecular and structural biology, which generate a vast amount of data, as exemplified by high-throughput DNA sequencing technology. Previously, the primary role of bioinformatics was to organize and manage these data; today, the major task of bioinformatics is to interpret the data with regard to various types of biological information. The data originally consisted largely of sequences of DNA and proteins and 3-D structures; now other types of data are becoming available, such as gene expression profiles generated by DNA chip technologies and 2-D protein maps of various cell and tissue types. At the same time, there is a huge body of knowledge on molecular interactions and biochemical pathways that exists only in the literature or in the minds of experts; this knowledge has to be computerized, organized and analyzed in order to transform the biological data into useful information for academic and industrial research (Persidis, 1999).

Two aspects of bioinformatics are relevant for data interpretation on a massive scale: development of new algorithms and software, and computerization of data and knowledge. For example, although it is important to develop rapid and sensitive methods for searching through databases for related sequences of biological materials, interpretation of the results would not be possible without the related biological information which is associated with the sequences stored in the database (Frishman, 1998).

The use of a single-letter code for the description of molecular modular components, as well as the construction of complex molecular structures through linear sequences of such single-letter code representations, enables the description of complex modular biomolecules, such as DNA and proteins, as a linear sequence of letters. Such a linear sequence is the basis for storage, management and analysis of information related to those molecules. The basic DNA of every living organism can be described with a code of four letters. Each letter represents a chemical base found within a DNA molecule—Adenine (A), Thymine (T), Guanine (G) and Cytosine (C). Proteins and peptides are described via a single-letter code that enables the expression of all twenty (20) amino acids (the “building blocks” of protein). This code designates all amino acids in a form which can be understood by persons who are not expert chemists—Argenine (A), Lysine (K), Proline (P), etc. The idea that the rich variety of life can be reduced to mere single-letter codes once seemed overwhelming, but is now fully accepted. This code enables the denotation of a DNA hexamer with 4096 different “words” and a peptide hexamer with 6.4*10⁷ different words (Davis, 2000).

All sequence data is compiled in large databases; these sequences will soon be followed by data collections on e.g. expression data, protein-protein interaction data, phenotypic data for mutants, etc. Straightforward access to data via the Internet means that a wealth of information is available. The topics included within the purview of bioinformatics range from retrieving and aligning DNA and protein sequences to predicting the structure and function of gene products. These common aspects of bioinformatics address a number of issues. For example, U.S. Pat. No. 6,023,659 describes a system for storing biomolecular sequence information according to protein function hierarchies, such that the data is retrieved both according to sequence and according to function, thereby providing more information about the sequence data than could be obtained simply from the sequences themselves.

Originally, bioinformatics was invented to describe the task of handling, presenting and analyzing large amounts of sequence data. Today, due to intense efforts in a number of large research centers throughout the world, data can be rather easily accessed by anyone via the Internet and World Wide Web servers (Thayer, 2000). As a result, the screening of these sequence databases to find sequence homologues of a particular gene is currently almost an everyday activity in most molecular biology labs. Such searches are performed, not only to find homologues within a species, but also to look for similar, so-called orthologous, genes in other organisms. The discovery of numerous such orthologous groups of genes provides excellent support for the power of using of model organisms. Sequence similarity is also used to cluster organisms according to their evolutionary affinity, and thus to create phylogenetic trees, an important tool in taxonomy. In parallel to the DNA sequencing effort, determination of the location of genes on chromosomes is today performed in large-scale projects for a number of organisms, which provide information that needs to be efficiently handled and presented (Abbott, 1999).

These linear sequences are then preferably employed for three-dimensional structural prediction. For most macromolecules, their function is closely linked to the three-dimensional structure; this is perhaps most apparent for proteins, DNA molecules and RNA molecules. Recent technical developments can now provide a more detailed view of how molecules are folded. The experimental determination of these three-dimensional structures, however, is a costly and slow process. Novel procedures for predicting the molecular folding from the primary sequence data are therefore urgently needed. Since the protein structure ultimately carries the information on the enzymatic active site or surface site for protein-protein interaction, knowledge of the protein tertiary structure will be of fundamental importance for the pharmaceutical industry in future (Searls, 2000; Rawlings, 1997).

Such analyses of the sequence and predicted structure of individual molecules is currently being extended to analysis of genome-wide biomedical data and functional genomics (Gotoh, 1999; Searls, 2000).

In the last few years, the advent of large-scale biomedical analysis tools have irreversibly changed research procedures for scientists in the fields of biology and medicine. These technologies enable the simultaneous study of the expressions of thousands of genes, at either the transcript or the protein level, or of the thousands of possible protein-protein interactions in a cell, or phenotypic analysis of thousands of mutants etc. All of these data, regardless of type and format, must be handled, presented and efficiently analyzed. This challenge is already being explored by statisticians for the clustering of e.g. similarly regulated genes. This clustering information is currently being evaluated as a potentially useful way of predicting the function of functionally uncharacterized genes in the follow-up on the genomics projects—a research area known as functional genomics. The prediction of gene function may eventually include more complex procedures, such as the integrated analysis of many types of large-scale molecular data into one tentative function for the studied gene. This latter task will, of course, also utilize information gained by applying the above described sequence analysis. In addition, genes with similar expression profiles would possibly exhibit common sequence elements in their regulatory regions. Identifying these sequences by means of computerized methods, which is more difficult than finding clear similarities between the encoded proteins, will be a great challenge that can provide extremely useful information (Gotoh, 1999; Brazma, 1998).

Ultimately, these large-scale analyses may result in the mathematical modeling of life processes. The vast amounts of data generated by the genome-wide analytical technologies will not only have to be clustered, but also, and more importantly, to be interpreted in a physiological context. To enable this interpretation in a more sophisticated manner than is currently possible when handling thousands of information units, computerized strategies will have to be developed. This is a formidable task that incorporates modeling of all molecular processes in a cell at the molecular level. Initially, this task will be approached by modeling of discrete parts of the cell physiology, such as metabolic fluxes or regulatory networks. However, the integration of all these will in many ways constitute the ultimate challenge for bioinformatics and an important part of the final goal of biomedical science in general—the complete molecular understanding of a living organism (Gershon, 1997).

In addition, regardless of the type of information which is to be generated, analyzed and finally interpreted, the data must be presented to the scientific community by establishing Internet-based World Wide Web servers. The presentation of this data can be rather challenging; the problems that may arise extend from the form of data submission to the need for intelligent and clear ways of presentation. Database management is thus not only an engineering problem, but also provides a clear scientific challenge, which is currently being addressed for protein and genetic material databases (Ouellette, 1999).

Glycoconjugates and Lectins

In addition to such well-known functions as structural and energy storage, carbohydrates (glycoproteins, proteoglycans and glycolipids) play a major role in most biological and pathological activities. Complex carbohydrates are essential in almost all forms of molecular recognition, as well as in processes involving fertilization, development of immune response, cell-cell communication and adhesion, inflammation, various cancers, central nervous system and autoimmune response, cardiovascular disease, diabetes and cellular invention of virus and bacteria. By “complex carbohydrate”, it is also meant oligosaccharide as well. The term “carbohydrate” includes complex carbohydrates, monosaccharides and oligosaccharides. Carbohydrates of biological relevance in the above areas usually consist of several covalently linked monosaccharide units and are referred to as complex carbohydrates or oligosaccharides and glycans. There are ten monosaccharides found in mammalian systems which may be additionally modified, typically by acylation or sulphation. Oligosaccharides are in most cases associated with other biomolecules, such as lipids or proteins; these hybrids, known as glycoconjugates, can be classified as glycoproteins, glycolipids and proteoglycans.

Glycoproteins are by far the most complex glycoconjugates and account for functions such as the determination of blood type. There are two major classes of glycoproteins, O-linked and N-linked, depending on whether the oligosaccharide chain is linked to the protein via threonine or serine side chains (O-linked) or via aspargine (N-linked). The oligosaccharide chains themselves are often branched, and a large number of sub-types exist.

Glycolipids are composed of an oligosaccharide covalently linked to a fatty acid portion by means of an inositol or sphingosine moiety. The association of the non-polar function with the cell membranes effectively anchors these molecules to the extracellular surface. One class of glycolipids, known as glycophosphatidyl inositol anchors, acts as sites of attachment for proteins to the cell membrane. Another type, the gangliosides, are thought to be crucial in the development of nervous tissue. The carbohydrate portion of glycoproteins and glycolipids often acts as a site for the binding of other large biomolecules, such as cell-surface proteins (called lectins or adhesins), bacterial toxins, hormones and antibodies. As such, glycoconjugates mediate many cell-cell interactions; they are not only responsible for the defense of an organism against pathogens, but also, paradoxically, often facilitate infection.

Lectins are multivalent carbohydrate-binding proteins which specifically bind (or crosslink) carbohydrates. By way of exception, ricin, the oldest lectin, is actually the enzyme RNA-N-glycosidase, Charcot-Leyden crystal protein (galectin-10) is known as lysophospholipase, and I-type lectins such as sialoadhesin are members of the immunoglobulin superfamily. Multivalency may not be an absolute requirement, even though it is still an important factor for most lectins. Since lectins generally have no apparent catalytic activity, as do enzymes, their physiological functions remain unclear. Unfortunately, for this reason, the term “lectin” has sometimes been used as a convenient taxon to “group out” carbohydrate-binding proteins, the functions of which were unknown.

Lectins are often classified on the basis of saccharide-specificity. Though this conventional method is familiar and useful in practice, it is not necessarily relevant for refined specificity. Lectins in the same category (e.g., galactose-specific lectins) show considerably different sugar-binding preferences. Moreover, an increasing number of lectins which never show high affinity to simple saccharides have been found.

From the standpoint of modern molecular biology, lectins should be understood as constituting protein families. However, during the projects to determine the sequence of the genomes of various organisms, including humans, the initial classification of lectins as protein (gene) families led to the realization that there are thousands of lectin genes waiting for functional decoding. Nevertheless, the above genetic approach is not enough to understand the essence of lectins. For example, even though members of the same families are similar, it does not necessarily mean they are the same (they usually have some degree of individual “personality”). The matter of “species specificity” is also involved. Thus, many general and specific features and characteristics of lectins remain unresolved.

Glycobioinformatics

In contrast to nucleic acids and proteins, whose primary structure is linear in nature, carbohydrates are branched molecules. It has been calculated that a carbohydrate hexamer may have 1.05×10¹² permutations (Laine, 1994). In addition to the branching complexity, the anomeric stereochemistry, ring size and subunit modifications of carbohydrates such as phosphorylation, sulphation, acetylation and many more show truth of the statement of Nathan Sharon in 1975 (“Complex Carbohydrates: Their Chemistry, Biosynthesis and Functions”, by Nathan Sharon, Addison-Wesley Publishing Company, Massachusetts, USA, 1975): “indeed, we know now that the specificity of many natural polymers is written in terms of sugar, not amino acids or nucleotides”. But this idea did not become pervasive until recently; as a result, sugar/saccharide/complex carbohydrate bioinformatics are lagging far behind DNA and peptide bioinformatics, and at times do not even exist. In spite of the abundance of carbohydrates in nature and their important role in many biological and pathological processes, glycobioinformatics remains an extremely limited discipline.

In particular, only a few groups have attempted to address some aspects of carbohydrate bioinformatics, such as carbohydrate modeling and three-dimensional structure (Bohne, 1998; Imberty, 199; Bush, 1999; Gohiet, 1996; Von der Lieth, 1998). For example, the CarbBank (Complex Carbohydrate Structure Database—CCSD) which includes 48,956 records which were derived from published articles and compiled by the Complex Carbohydrate Research Center (CCRC), represents complex carbohydrates in a graphical or schematic manner only. The database does not have any tools for carbohydrate analysis, similarity or comparison, which severely limits its utility. Furthermore, unlike the genetic and protein databases of GeneBank and SwissProt, the CCSD was active only between 1993 and 1995, and was closed in 1999 due to financial problems, poor information management architecture and its limited capacity for analysis.

Between 1995 and 1997, the first and only attempt to discuss carbohydrate bioinformatics was made by a group headed by Willett (Bruno, 1997). Willett and co-workers, relying on the data stored in the CCSD, implemented a carbohydrate imaging representation in the form of labeled graphs, in which the nodes and edges of a graph were used to denote the residues and the inter-residue linkages respectively. These graph representations were then searched by means of the subgraph isomorphism algorithm of Ullman (Ullman, 1976). It was demonstrated that this graph theory approach provided a precise way of searching carbohydrate structures in the CCSD. Nonetheless, even though this software algorithm supported searching, it lacked sophistication and the ability of the many high quality software tools and algorithms of gene or protein sequence analysis. Thus, clearly a higher quality software program, with associated database, is required for searching and retrieving carbohydrates from a database, on the basis of homology comparisons and/or other types of analyses, particularly when the many important biological functions of carbohydrates are considered.

The past decade has seen a renaissance in carbohydrate biology and chemistry. The advent of effective methods for characterizing the complex carbohydrate structures present on the surface of cells has spawned a new appreciation of the varied biological functions of these molecules (Dwek, 1996), as well as of new methods for the large-scale synthesis of carbohydrates. The involvement and importance of carbohydrates in most forms of life, on one hand, and the tremendous and relatively neglected body of information, on the other hand, illustrate the necessity of creating software tools that will facilitate glycobioinformatics.

In particular, these software tools would require a simple yet complete representation of carbohydrates, which would facilitate the comparison and analysis of such structures by computer. The complexity of carbohydrate structure—including branching, sugar modification, stereochemistry, anomer and different ring size—makes the use of the single-letter system adopted for nucleic acids and proteins impossible.

There is thus a need for, and it would be useful to have, software tools, including an associated database, for the storage, retrieval and standardized computer analysis of highly complex carbohydrate structures.

### SUMMARY OF THE INVENTION

The present invention is of a system and method for storing, retrieving, comparing and analyzing complex carbohydrates, by representing complex carbohydrates with a simple linear code, which is preferably also able to represent branches and modifications within the carbohydrate structure. The method of the present invention for converting the carbohydrate structure to such a linear code includes the steps of parsing each component of the structure; separately demarcating each branch within the structure; and then converting each component to a symbolic representation which may optionally be alphabetic, numeric, or a combination thereof.

According to the present invention, there is provided a method for representing a carbohydrate structure as a linear sequence, the steps of the method being performed by a data processor, the method comprising the steps of: (a) decomposing the carbohydrate structure into a plurality of elements; (b) determining a connection between each pair of elements; and (c) constructing a series of the plurality of elements connected with the connections to form the linear sequence.

According to another embodiment of the present invention, there is provided a method for comparing a first carbohydrate structure to a second carbohydrate structure, the steps of the method being performed by a data processor, the method comprising the steps of: (a) providing each of the first and the second carbohydrate structures as a first and second linear sequence, respectively; (b) comparing at least a portion of the first linear sequence to the second linear sequence to form a comparison; and (c) determining a homology score according to the comparison.

According to yet another embodiment of the present invention, there is provided a method for representing a post-translation modification of a protein, the steps of the method being performed by a data processor, the method comprising the steps of: (a) providing a linear code for describing carbohydrate structures; and (b) representing the post-translation modification as a linear sequence with the linear code.

By “complex carbohydrate”, it is also meant oligosaccharide as well. The term “carbohydrate” includes complex carbohydrates, monosaccharides and oligosaccharides.

Hereinafter, the term “computational device” includes, but is not limited to, personal computers (PC) having an operating system such as DOS, Windows™, OS/2™ or Linux; Macintosh™ computers; computers having JAVA™-OS as the operating system; graphical workstations such as the computers of Sun Microsystems™ and Silicon Graphics™, and other computers having some version of the UNIX operating system such as AIX™ or SOLARIS™ of Sun Microsystems™; or any other known and available operating system, or any device, including but not limited to: laptops, hand-held computers, PDA (personal data assistant) devices, cellular telephones, any type of WAP (wireless application protocol) enabled device, any type of device which operates according to the Bluetooth standard or any other wireless standard, wearable computers of any sort, which can be connected to a network as previously defined and which has an operating system. Hereinafter, the term “Windows™” includes but is not limited to Windows95™, Windows 3.x™ in which “x” is an integer such as “1”, Windows NT™, Windows98™, Windows CE™, Windows2000™, and any upgraded versions of these operating systems by Microsoft Corp. (USA).

For the present invention, a software application could be written in substantially any suitable programming language, which could easily be selected by one of ordinary skill in the art. The programming language chosen should be compatible with the computational device according to which the software application is executed. Examples of suitable programming languages include, but are not limited to, C, C++, Perl and Java.

In addition, the present invention could be implemented as software, firmware or hardware, or as a combination thereof. For any of these implementations, the functional steps performed by the method could be described as a plurality of instructions performed by a data processor.

### BRIEF DESCRIPTION OF THE DRAWINGS

The invention is herein described, by way of example only, with reference to the accompanying drawings, wherein:

FIG. 1 is a flowchart of an exemplary method for performing a sequence homology comparison and analysis according to the present invention;

FIG. 2 is a flowchart of a particular illustrative method for comparing the sequences according to the present invention; and

FIG. 3 is a schematic block diagram of an exemplary system according to the present invention for carboydrate sequence analysis.

### DESCRIPTION OF THE PREFERRED EMBODIMENTS

The present invention is of a set of software tools, including an associated database, for storing, retrieving, comparing and analyzing complex carbohydrates. These software tools rely upon the representation of even complex carbohydrates with a simple linear code, which is preferably also able to represent branches and modifications within the carbohydrate structure. The method of the present invention for converting the carbohydrate structure to such a linear code includes the steps of parsing each component of the structure; separately demarcating each branch within the structure; and then converting each component to a symbolic representation which may optionally be alphabetic, numeric, or a combination thereof.

In order to meet the requirements for such a simple linear code, the present invention provides a multi-letter code, composed of units described with regard to a Saccharide Unit (SU) letter code. The SU describes, as a linear string, all physical parameters expressing the carbohydrate parameters, while the syntax expresses the way the carbohydrate connected to each other, preferably including the branches.

Once the structure of the carbohydrate has been rendered as a linear code, these linear codes may optionally and preferably be compared. The method of the present invention for comparing these linear codes is preferably performed as follows. Briefly, the query and target carbohydrate structures are entered for comparison, preferably already as the linear code sequence. These sequences are then divided into saccharide units. Although any type of string comparison algorithm which is known in the art could be used, preferably the sequences are compared by “sliding” the query sequence against the target sequence, resulting in a comparison of each saccharide unit and each sub-sequence of saccharide units of the query and target complex carbohydrates. The results of this comparison procedure are then analyzed in order to determine the homology score.

Potential applications for the methods of the present invention include, but are not limited to, the management of carbohydrate databases, and searching through such databases in order to find and retrieve sequences of interest which are identical or similar to a query sequence; drug discovery, for example through the identification of biosynthetic pathways and inhibitors; comparative analysis; functional identification of newly discovered carbohydrate structures through a comparison to carbohydrates having known functions; functional identification of protein sequences having an unknown structure, which may be expected to bind to a carbohydrate sequence having an unknown structure; and to describe the in vitro synthetic pathways for carbohydrate structures. For both in vitro and in vivo synthetic pathways, the method of the present invention could optionally be used to describe these pathways as a set of linear equations, with participating carbohydrate structures being represented with linear sequences in the linear code.

Another application for the methods of the present invention is to describe glycosylation as a post-translation modification of proteins with the linear code. For example, if a protein receives such a post-translation modification in the form of an added complex carbohydrate structure, this complex carbohydrate structure could be described with the linear code, thereby enabling the glycosylation to be stored in the database, along with the protein sequence. Such a complex carbohydrate structure could even optionally be searched and retrieved with a query sequence, for example in order to locate homologous post-translation modifications of proteins. Examples of suitable protein databases for storing such added linear code sequences include, but are not limited to, SwissProt and PDB (Brookhaven Protein Databank).

The carbohydrate linear code of the present invention digitizes the last analog data in biological science and opens a vast potential in bioinformatics, drug discovery and Web applications. The location of similarities and homologies between carbohydrate structures and the compilation of the entire bio-relevant information package will open another frontier for the drug discovery science and industry. Even though human lectins are of great importance in major biological and pathological processes, most lectins, their genes and their exact functions are not known. Comparison of known lectin genes in terms of the carbohydrate structure bound by these proteins would support the search for similar carbohydrate structures, as well as the identification of new lectin genes and evaluation of their potential function. The new opportunities provided by this novel linear code have opened a new era in discovery of glyco-related drugs and targets, whether carbohydrates, proteins or a combination thereof.

The following description is divided into sections, in order to further facilitate the discussion of the different elements of the present invention for the storage, retrieval, comparison and analysis of complex carbohydrates. The first section, entitled “Linear Code Syntax”, discusses the linear code itself; the second section, entitled “Method of Analysis”, describes an exemplary method of analysis and comparison according to the present invention; the third section, entitled “Comparison Scores for Each Saccharide Unit”, describes an exemplary specific method for comparing pairs of saccharide units; the fourth section, entitled “Comparison of Junctions”, describes an exemplary specific method for comparing pairs of junctions; the fifth section, entitled “Further Analysis of Similarity Elements”, describes an exemplary specific method for defining clusters of similar saccharide units; the sixth section, entitled “Specific Example of Analysis Method”, describes a specific overall example of the operation of the method of the present invention; and the seventh section, entitled “Exemplary System for Sequence Analysis”, describes an exemplary system according to the present invention.

Section 1: Linear Code Syntax

The syntax of the linear code of the present invention requires the components of the carbohydrate to be represented as simple, repetitive elements. Collectively, these elements form the linear code, which is capable of representing even complex carbohydrate structures as simple linear sequences. According to the present invention, each such repetitive element is termed herein a “basic saccharide unit”.

The basic Saccharide Unit (SU) is composed of five parts: the sugar name, any modifications to the sugar, the anomer, the position according to which the sugar is connected to the neighboring sugar, and the presence of a branch (if any).

Sugar name—The sugar name is represented by one capital letter, and is determined by a monosaccharide name table, an example of which is given below.

| Trivial Name | Monosaccharide/Core | Linear Code |
|---|---|---|
| D-Galp | D-Galactose pyranose | A |
| L-Galp | L-Galactose pyranose | A′ |
| D-Galf | D-Galactose furanose | A! |
| L-Galf | L-Galactose furanose | A˜ |
| D-GalpNAc | N-Acetyl-D-Galactosamine | AN |
| D-GalpN | deacetylated D-GalpNAc | AQ |
| D-Ribp | D-Ribose pyranose | B |
| Cer | Ceramide | C |
| Sph | Sphingoside | D |
| L-Fucp | L-Fucose pyranose | F |
| D-Glcp | D-Glucose pyranose | G |
| D-GlcpNAc | N-Acetyl-D-Glucosamine | GN |
| L-Rhap | L-Rhamnose pyranose | H |
| D-Idop | D-Iodoronic acid | I |
| KDN | 2-keto-3-deoxynananic acid | K |
| D-GalpA | D-Galactoronic acid | L |
| D-Manp | D-Mannose pyranose | M |
| D-Neu | Neuraminic acid | N |
| D-Neu5G | 5-N-glycolyl neuraminic acid | NJ |
| D-Neu5Ac | 5-N-acetyl neuraminic acid | NN |
| L-Araf | L-Arabinose uranose | R |
| D-GlcpA | D-Glucoronic acid | U |
| D-Xylp | D-Xylose pyranose | X |
| | Polymer | Z |

The following abbreviations should be noted:

MS′=Opposite stereospecificity to the common structure D< >L

MS!=Opposite structure to the common structure P< >F

MS˜=Rare sugar with double opposite both in stereospecificity and in structure.

Not all possible sugars in nature are described, even in this more complete table. For example, there are many less common sugars that are less relevant to carbohydrates found in mammalian species (Xylulose, Erythrulose, Tagose and so forth). However, this example clearly demonstrates that the table code easily permits the addition of any desired sugar by adding unique capital letters.

Sugar modifications—The modifications are represented by brackets (the “[”, “]” characters) with number-and-letter pairs inside them. The number denotes the position of the modification; the letter denotes the modification itself, determined by the modification table, an example of which is given below.

| Modification Type | Symbol |
|---|---|
| anhydrous | Y |
| hydroxyl | OH |
| Alcohol | O |
| Pyruvate | V |
| Sulfate | S |
| Sulfide | SH |
| Phosphate | P |
| Aminoethylphosphate | PN |
| Ethanolaminephosphate | PO |
| Choline | CH |
| deacetylated N-Acetyl | Q |
| N-glycolyl | J |
| O-Lactyl | LL |
| N-Acetyl | N |
| O-Acetyl | T |
| O-Methyl | E |
| Cholesterol | CHO |
| Carboxylate | OOH |

It should be noted that only common modifications appear in the above exemplary table. A large number of alkyl groups, such as: ethyl, propyl, butyl, pentyl, hexyl, heptyl and many more, are not represented here, as well as a large number of acyl groups, such as: numerate, acetate, propaoate, butanoate and more. These modifications could certainly be added if desired. Other modifications might optionally be synthesized onto sugar molecules; as such, any modification can be added, but with a unique code.

In addition, certain common modifications can be written as an appendix to the sugar name, for example : A[2S]→AS. The rules for the common modifications are preferably included in a modification translation table, an example of which is given below as a list:

AQ=A[2Q]

AN=A[2N]

GN=G[2N]

NN=N[5N]

NJ=N[5J]

Anomer—The anomer appears after the modifications, if any such modification is present, and is denoted by the letter “a” (representing the α-anomer) or “b” (representing the β-anomer).

Position—The position at which the sugar is connected to the neighboring sugar is represented by a number, and appears last in the SU.

The following table gives a number of examples of the basic saccharide unit, with various types of modifications, and so forth, with accompanying notes.

Basic Sugar Names

| Sugar | Sugar | | | |
|---|---|---|---|---|
| Name | Modifications | Anomer | Position | Notes |
| A | | b | 3 | No modifications |
| A | [3S] | a | 5 | One modification |
| G | [2S3Q] | b | 3 | Two modifications |
| NN | | a | 4 | One common modification, |
| | | | | written next to the sugar name |
| AN | [3Q] | a | 2 | Two modifications, one |
| | | | | commonly written next to the |
| | | | | sugar name and the second in |
| | | | | brackets |

The power of the simple linear code of the present invention is demonstrated in the following example. Sialic acid is an acidic sugar with many modifications, yet the linear code enables them to be easily written as follows.

| Linear Code | Trivial name | Nomenclature |
|---|---|---|
| N | Neu | Neuruminic acid |
| NN | Neu5Ac | N-Acetylneuruminic acid |
| NN[4T] | Neu4,5Ac2 | N-Acetyl-4-O-acetylneuruminic acid |
| NN[7T] | Neu5,7Ac2 | N-Acetyl-7-O-acetylneuruminic acid |
| NN[8T] | Neu5,8Ac2 | N-Acetyl-8-O-acetylneuruminic acid |
| NN[9T] | Neu5,9Ac2 | N-Acetyl-9-O-acetylneuruminic acid |
| NN[4T9T] | Neu4,5,9Ac3 | N-Acetyl-4,9-di-O-acetylneuruminic acid |
| NN[7T9T] | Neu5,7,9Ac3 | N-Acetyl-7,9-di-O-acetylneuruminic acid |
| NN[8T9T] | Neu5,7,8,9Ac4 | N-Acetyl-8,9-di-O-acetylneuruminic acid |
| NN[7T8T9T] | Neu5,8,9Ac4 | N-Acetyl-7,8,9-tri-O-acetylneuruminic acid |
| NN[9L] | Neu5Ac9Lt | N-Acetyl-9-O-lactylneuruminic acid |
| NN[4T9L] | Neu4,5Ac2,9Lt | N-Acetyl-4-O-acethyl-9-O-lactylneuruminic acid |
| NN[8E] | Neu5Ac8Me | N-Acetyl-8-O-methylneuruminic acid |
| NN[8E9T] | Neu5,9Ac2,8Me | N-Acetyl-9-O-acetyl-8-O-methylneuruminic acid |
| NN[8S] | Ncu5Ac8S | N-Acetyl-9-O-phosphoroneuruminic acid |
| NN[9P] | Neu5Ac9P | N-Acetyl-8-O-sulphoneuruminic acid |
| NN[2Y7Y] | Neu2,7an5Ac | 5-N-acetyl-2,7-Anhydro-neuruminic acid |
| NJ | Neu5Gc | N-Glycolyl-neuruminic acid |
| NJ[4T] | Neu4Ac5Gc | 4-O-Acetyl-5-N-glycolyl-neuruminic acid |
| NJ[7T] | Neu7Ac5Gc | 7-O-Acetyl-5-N-glycolyl-neuruminic acid |
| NJ[8T] | Neu8Ac5Gc | 8-O-Acetyl-5-N-glycolyl-neuruminic acid |
| NJ[9T] | Neu9Ac5Gc | 9-O-Acetyl-5-N-glycolyl-neuruminic acid |
| NJ[7T9T] | Neu7,9Ac2,5Gc | 7,9-Di-O-Acetyl-5-N-glycolyl-neuruminic acid |
| NJ[8T9T] | Neu8,9Ac2,5Gc | 7,8,9-Tri-O-Acetyl-5-N-glycolyl-neuruminic acid |
| NJ[7T8T9T] | Neu7,8,9Ac3,5Gc | 8,9-Di-O-Acetyl-5-N-glycolyl-neuruminic acid |
| NJ[9L] | Neu5Gc9Lt | 5-N-Glycolyl-9-O-lactyl-neuruminic acid |
| NJ[8E] | Neu5Gc8Me | 5-N-Glycolyl-8-O-methyl-neuruminic acid |
| NJ[8E9T] | Neu9Ac5Gc8Me | 9-O-Acetl-5-N-glycolyl-8-O-methyl-neuruminic acid |
| NJ[7T8E9T] | Neu7,9Ac2,5Gc8Me | 7,9-Di-O-acetl-5-N-glycolyl-8-O-methyl-neuruminic acid |
| NJ[8S] | Neu5Gc8S | 5-N-Glycolyl-8-O-sulpho-neuruminic acid |
| NNJ | Neu5GcAc | N-(O-Acetyl)glycolyl-neuruminic acid |
| NJE | Neu5GcMe | N-(O-Methyl)glycolyl-neuruminic acid |
| NJ[2Y7Y] | Neu2,7an5Gc8Me | 2,7-Anhydro-5-N-glycolylneuruminic acid |
| NJ[2Y7Y8E] | Neu2,7an5Gc | 2,7-Anhydro-5-N-glycoly-8-O-methylneuruminic acid |
| K | Kdn | 2-Keto-3-deoxynononic acid |
| K[9T] | Kdn9Ac | 9-O-Acetyl-2-Keto-3-deoxynononic acid |

Complex Carbohydrates (CC's)

The basic saccharide unit is then used to build each complex carbohydrate, which is constructed of a plurality of linked saccharide units (SU). The CC is written such that the saccharide units are arranged from right to left, such as Aa2Ga4Mb3 for example. The last character at the right may optionally be a conjugate. For example, “C” could be ceramide, “P” could be polymer, and so forth.

When written in terms of trivial names, or with a regular graphic or schematic representation, a complex carbohydrate which features six monosaccharides would look like this:

β-D-Galp-(1→4)-β-D-GlcpNAc-(1→3)-β-D-Galp-(1→4)-β-D-GlcpNAc-(1→3)-β-D-Galp-(1→4)-β-D-Glcp-(1→1)-Ceramide

In the linear code of the present invention, the same structure is written as follows:

Ab4GNb3Ab4GNb3Ab4GbC

Clearly, the latter representation is far simpler to write, store, retrieve and to compare to other carbohydrate sequences. Many string comparison tools exist, for example for the purpose of performing homology comparisons for genetic material such as DNA sequences. Thus, the reduction of a lengthy complex carbohydrate description to a simple linear string demonstrates the clear advantage of the linear code of the present invention even for linear complex carbohydrates.

Branched CC

A more complex case is created when the carbohydrate structure features one or more branches. Unlike other types of biological materials, such as DNA and proteins for example, which feature simple linear sequences of their basic elements, carbohydrates may have branched structures. Such branched structures are preferably handled by the simple linear code of the present invention such that the linearity of the represented sequences is maintained.

Branches are optionally and preferably represented by parentheses (the “(“,”)“characters). An open-parenthesis character appears at the beginning of each branch and a closed-parenthesis character at its end.

The decision as to which node appears within the parentheses and which appears outside of the parentheses is more preferably based on the first SU of each node. Optionally and preferably, the assignment of a portion of the sequence to be either outside or within the parentheses is implemented as follows.

First, if the saccharide units have different sugar names, the monosaccharide hierarchy for branching table is preferably examined. The hierarchy is written from the most to the least branched monosaccharide, meaning that monosaccharides which appear in a higher place in the table are preferably placed into parentheses first. The sugar in the hierarchy is written as an absolute value, without considering if it is in D or L form, or if it is pyranose or furanose. An example of this table, with accompanying explanation, is shown below.

| | Linear Code |
|---|---|
| Trivial Name | Hierarchy |
| Arabinose | R |
| Ribose | B |
| Xylose | X |
| Fucose | F |
| Rhamnose | H |
| Iodoronic acid | I |
| Galactoronic acid | L |
| Glucoronic acid | U |
| Neuraminic acid | N |
| N-acetyl Neuraminic acid | NN |
| Mannose | M |
| N-acetyl Galactoseamine | AN |
| N-acetyl Glucoseamine | GN |
| Galactose | A |
| Glucose | G |

This table contains the hierarchy which determines the relative location of a portion of the sequence as belonging inside or outside the parentheses. This hierarchy is more preferably empirically determined according to the frequency with which certain sugars appear at the branch node, in order to minimize the amount of the sequence which is placed within the parentheses.

If the units have the same sugar name, their positions are examined. The saccharide unit with the larger position number is preferably written within the parentheses.

For example, a complex carbohydrate structure that includes one branch such as ganglioside GM1 is written as follows:

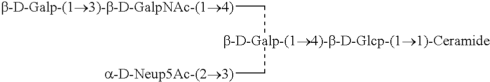

According to the steps described above, the sugars D-GalpNac and D-Neup5Ac are preferably compared according to the information which is stored in the hierarchy table; since D-Neup5Ac is at the higher hierarchy in the table, this sugar is then written within the parentheses. The linear code format of the above branched structure is: Ab3ANb4(NNa3)Ab4GbC

Another example in which the same sugar type is found at a branch point is demonstrated by the following structure:

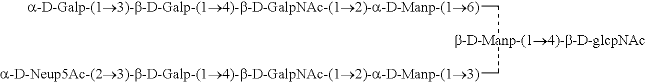

In this structure, according to the linear code, for a monosaccharide from a pair of otherwise identical sugars, the monosaccharide with the larger position number is placed within the parentheses. Therefore, the structure is expressed as: NNa3Ab4GNb2Ma3(Aa3Aa4GNb2Ma6)Mb4GN

Complex carbohydrates may have multiple branches. For example, a compound that includes several branches, each starting with a different sugar type, is commonly represented as:

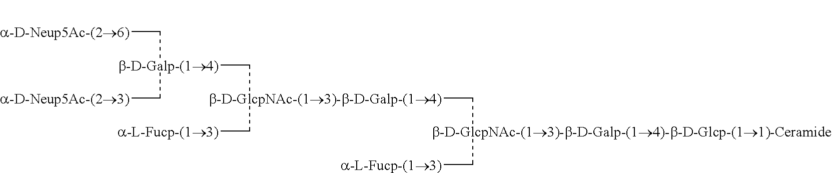

In linear code of the present invention, this structure is written as:

NNab3(NNa6)Ab4(Fa3)GNb3Ab4(Fa3)GNb3Ab4GbC

Certain carbohydrate structures may also have nested branches, which can be represented by the linear code of the present invention as well, simply by specifying the open parentheses each time a new branch starts. For example, the common graphical or schematic written form for the following complex carbohydrate is typically written as follows:

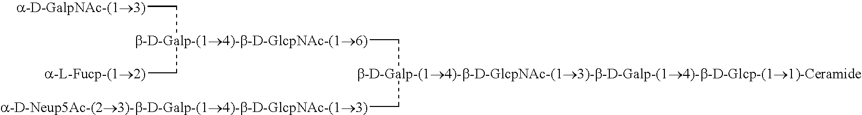

The structure for this complex carbohydrate is fully described by the linear code of the present invention as follows:

NNa3Ab4GNb3(ANa3(Fa2)Ab4GNb6)Ab4GNb3Ab4GbC

The linear code of the present invention is highly versatile and enables expression of highly branched structures with extreme ease. For example, triple branch points are other complex structures are fully described by the linear code of the present invention in a predictable and reproducible manner.

A triple branched junction exists in nature as well and is easily described by the linear code. Contiguous brackets opened one after the another show that in this node, several child nodes are present. For example, the complex structure:

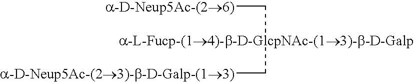

is expressed in the linear code of the present invention as:

NNa3Ab3 (NNa6)(Fa4)GNb3A

The following example shows the operation of multiple rules for determining the linear code for the carbohydrate structure. The highly complex structure of the following carbohydrate is graphically or schematically written as follows:

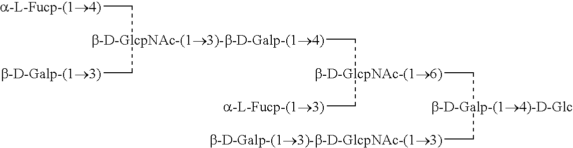

The linear code of this structure is:

Ab3GNb3(Ab3(Fa4)GNb3Ab4(Fa3)GNb3)Ab4G

As another example, polysaccharides are composed of a plurality of repeated carbohydrate units. Such polysaccharides are optionally represented with the basic repeated unit contained in curly brackets, or “{” and “}”, followed by the number of repetitions of the basic unit.

Certain types of components are more difficult to describe within the linear code of the present invention, including doubles, unknown elements and wildcard elements. For example, there may optionally be one or more components in a SU or in a CC which are unknown. These components are preferably written as follows. First, if only one of the components of a SU is unknown, the “?” character is used For example, for the linear code:

AN?3

the anomer type (a/b) is unknown.

For this linear code:

ANb??b4

the position of the left SU and the sugar name of the right SU are unknown.

For the next linear code:

A[?T7?]a3

the SU has a modification, but the position of the T and the identity of the 7 position are unknown.

There can optionally be a combination of as many unknown components as needed, such as for the following linear code:

A[?T]???[5?]a3.

However, if an entire SU in the CC is unknown, the “*” character is preferably used. For the linear code:

ANb3*Ab4

there are 3 saccharide units, but the identity of the middle SU is unknown. This is identical to writing the following linear code:

ANb3???Ab4.

However, it should be noted that the “?” character preferably replaces one component, and not one character, such that for example, the sugar AN is replaced by “?” and not by“??”.

In addition, a combination of such characters can optionally be used, as in the following linear code:

ANb?*Ga4M[?T]?3

which states that the anomer and the position of the modification of the first SU are unknown. The entire third SU is also unknown, and so is the position of the fourth SU modification, as well as the identity of the entire SU itself.

Another type of character which can be used to represent a structure with a degree of indeterminacy is the doubles characters. These characters are useful when the user is not certain of the identity of a particular SU or CC, but does not want to use the symbol for an unknown SU or CC. The doubles character is used to insert a CC which has several meanings. This can be done with the “/” character.

The “/” character could be used, for example, when entering a new CC into the database of carbohydrate sequences, such that the new CC is determined to have one of a limited number of identities. For example, the doubles character could be used as follows:

ANb3/4

which means that the position can only be 3 or 4, but nothing else.

The “/” character may optionally be used several times in one CC. For example, the linear code:

AN3/4G/Fa/b5N[3/4G]b7

may be rewritten to emphasize the meaning of what each “/” denotes:

AN3/4 G/Fa/b5 N[3/4G]b7.

Altogether, this CC can be interpreted in (2\hat{ }4=)16 different ways!

Optionally and more preferably, although any number of “/” characters may be written for a SU or a CC, no more than two values may be entered for each “/”. Therefore, the linear code:

ANa/b3/4

is allowed, but the linear code:

ANa3/4/5

is not allowed.

One of two saccharide units may be selected for this type of representation as a possible element, as an entire SU, with the “//” symbol. For example, the linear code:

Aa3//Gb2

states that one of the two monosaccharides is the correct monosaccharide.

Combinations of these different unknown elements are preferably possible. For example, the linear code:

Aa/b4//Ga2/3

is interpreted to mean that one of the following possible options (Aa4, Ab4, Ga2, Ga3) is true, although the identity of the correct element is not known. This notation more preferably prompts the reader to select one or more of those SU's.

For the method of comparison of the present invention, doubles can be compared to all CC's which can be interpreted from this CC, and which have been approved by the user who entered this CC initially. Each such possible CC is preferably considered to be a regular CC for the purposes of homology comparison, for example. In other words, if a match is found with one of the components which previously constituted part of a double, this match is a legal match. Such multiple comparisons are clearly more difficult for the unknown elements, and therefore are preferably not performed. Instead, the unknown element preferably acts only as a space holder within the structure.

Double character symbols can also optionally and preferably be used to examine a comparison between a CC entered by the user and the CC's in the database. All of the rules which apply to the previous use of the double characters preferably apply to this case as well, except for a single change, which is the user can enter as many values as desired for the same component This means that the user can now write the linear code:

A[3N/T]a4/5/6Ga2//Ga3//Fb2

which would preferably interpreted as these 18 CC's—

A[3N]a4 preceded by Ga2 or Ga3 or Fb2

A[3N]a5 preceded by Ga2 or Ga3 or Fb2

A[3N]a6 preceded by Ga2 or Ga3 or Fb2

A[3T]a4 preceded by Ga2 or Ga3 or Fb2

A[3T]a5 preceded by Ga2 or Ga3 or Fb2

A[3T]a6 preceded by Ga2 or Ga3 or Fb2

The above CC can optionally be shortened even more, by writing the following linear code:

A[3N/T]a4/5/6Ga2/3//Fb2

which adds an internal double inside a SU that is itself part of a double. The user is again preferably asked to choose which one (or more) of these CC's should be used in running the comparison.

Of course, for complex carbohydrates which contain both double characters and branches, the syntax may be changed dramatically according to the identity of the element which fills the space designated by the double character.

For example, the linear code:

Gb4(Gb3/6)Fa3

would preferably be interpreted as these two CC's:

Gb4(Gb6)Fa3

Gb3(Gb4)Fa3

The main node is Gb4 for the first CC, but Gb3 in the second CC. Preferably, the system handles such changes dynamically while building the interpreted CC's.

The following examples illustrate the use of wildcard and doubles. The following graphical or schematic representation and linear code demonstrate how to write a CC when the modification position is not known. The graphical or schematic representation is:

Acetate-(1→?)-α-D-Neup5Ac-(2→8)-α-D-Neup5Ac-(2→3)-β-D-Galp-(1→4)-β-D-Glcp-(1→3)-β-D-Galp-(1→4)-β-D-Glcp-(1→1)-Ceramide

In the linear code, the structure is expressed as:

NN[?OOH]a8NNa3Ab4Gb3Ab4GbC

As another example, following graphical or schematic representation and linear code demonstrate how to write a structure when the glycoside bond is one of two possibilities. The graphical or schematic representation is as follows:

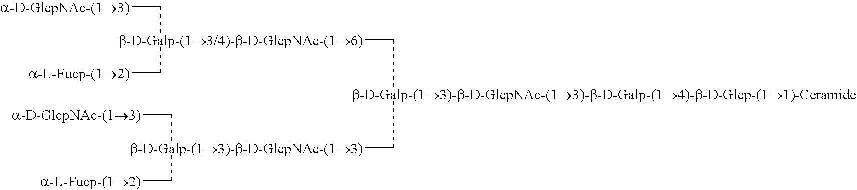

In the linear code, the structure is expressed as:

ANa3(Fa2)Ab4GNb3(ANa3(Fa2)Ab3/4GNb6)Ab3GNb3Ab4GbC

The following graphical or schematic representation and linear code demonstrate how to write a structure when the bond position is not known. The graphical or schematic representation is as follows:

α-D-Neup5Ac-(2→?)-α-D-Neup5Ac-(2→3)-β-D-Galp-(1→4)-β-D-Glcp-(1→1)-Ceramide

In the linear code, the structure is expressed as:

NNa?NNa3Ab4GbC

The following graphical or schematic representation and linear code demonstrate how to write a structure when the anomer is not known. The graphical or schematic representation is as follows:

?-D-GalpNAc-(1→3)-?-D-Galp-(1→4)-α-D-Galp-(1→4)-β-D-Galp-(1→1)-Ceramide

In the linear code, the structure is expressed as:

AN?3A?4Ab4C

Section 2: Method of Analysis

This section describes an exemplary method of analysis according to the present invention, for example for performing homology comparisons between two or more sequences which are written in the linear code of the present invention.

This method is preferably implemented as a software application, or other type of implementation as previously described, which assess homologies between complex carbohydrate (CC) sequences. The method is designed to assess the homology between a sequence which is entered or selected by the user and sequences in a database, in order to find, present and score the most homologous sequences. The determination of homologous string and structural elements in Linear Code is a powerful tool for clarification of the function and synthesis pathway of a new sequence by comparing its linear code to the codes of sequences with known or partly known function.

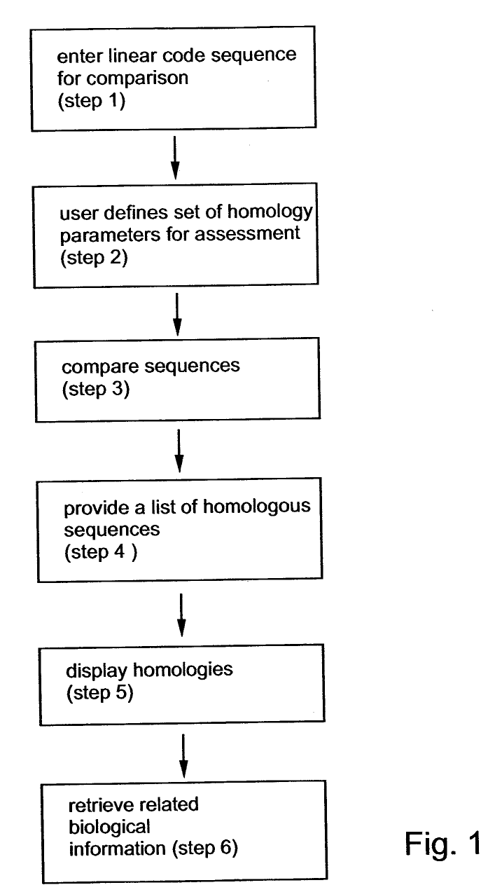

The following method is described with regard to the flowchart of FIG. 1. In step 1, the user preferably enters the linear code for a sequence to be compared. The term “entering” a sequence may optionally include selecting the sequence from a list of such sequences, as well as by manually entering the sequence by the user. Also optionally and preferably, the linear sequence could be automatically converted and/or translated from a known carbohydrate structure representation format.

The comparison is optionally performed against another such sequence which is entered by the user; against a plurality of such sequences which are stored in a database; or against a model of a theoretical carbohydrate structure which has been rendered in the linear code of the present invention.

In step 2, the user defines a set of parameters for homology analysis. These parameters may optionally change according to the biological function and similarities in which the user is interested. The user is preferably able either to use a preset combination of parameters optimized for a specific kind of query, or to set the value of the parameters according to the particular search to be performed.

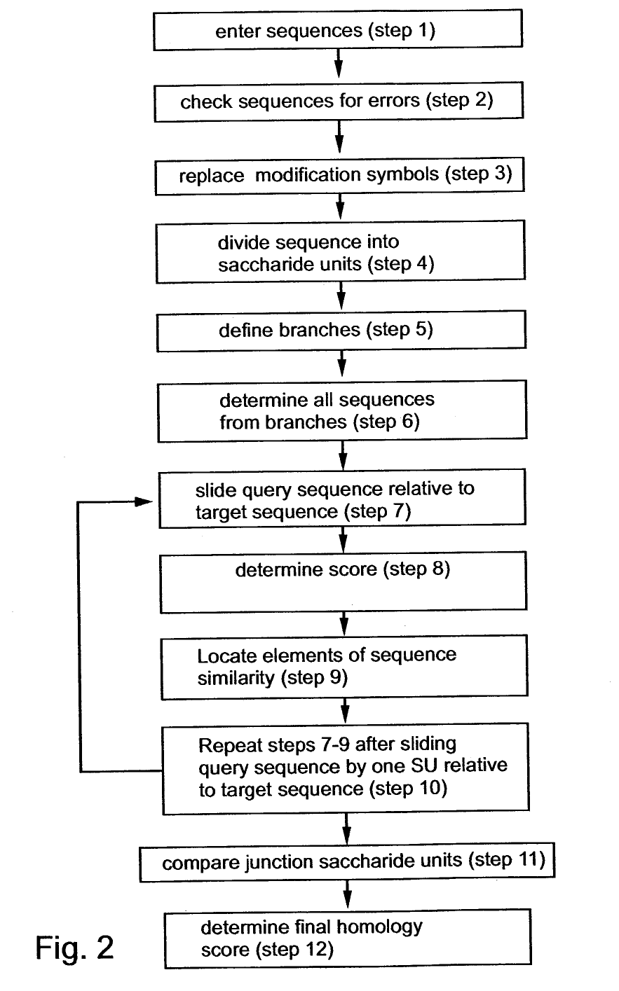

In step 3, the comparison, optionally with an accompanying search through a plurality of sequences is performed according to the method described in FIG. 2. The comparison preferably results in a numeric homology score.

In step 4, the output for the query is a list of CC's. Preferably, the final homology score for these CC's is above a certain threshold, and the CC's are listed according to their homology value. The homology score and the probability of finding a linear code with the same homology or higher by chance in the database is more preferably indicated for each CC that passes the threshold.

In step 5, if the user selects one of the homologous CC's, the user is more preferably able to see both the query and the target CC, with the elements of similarity highlighted according to degree of similarity.

In step 6, the user most preferably is able to retrieve additional biological and structural data related to the homologous CC's from the database.

As explained with regard to FIG. 2, the analysis method of the present invention involves a number of steps. Briefly, the query and target carbohydrate structures are entered for comparison, preferably already as the linear code sequence. These sequences are then divided into saccharide units. Although any type of string comparison algorithm which is known in the art could be used, preferably the sequences are compared by “sliding” the query sequence against the target sequence, resulting in a comparison of each saccharide unit and each sub-sequence of saccharide units of the query and target complex carbohydrates. The results of this comparison procedure are then analyzed in order to determine the homology score.

These steps are explained in greater detail with regard to the flowchart of the method shown in FIG. 2. As shown, in step 1, the query and target complex carbohydrate structures are entered for comparison, preferably in the linear code format of the present invention. If these carbohydrate structures are entered in a different format, such as the graphical or schematic format described previously, then these structures are first converted to the linear code of the present invention. The various formats for entering the linear code sequences are described with regard to FIG. 1.

In step 2, the syntax of the query sequence is examined for errors and/or illegal code elements. If any errors are found, then these are displayed to the user for correction.

In step 3, optionally and preferably, one or more modification symbols are replaced with the full name of the modification or the repeating components. Most preferably, the replacement is selected from the previously described modification interpretation table.

In step 4, the complex carbohydrate string is divided into the corresponding saccharide units (SU). Preferably, this process includes the steps of defining the beginning and ending of each SU, as well as the corresponding serial number, or position number of the saccharide unit, in the sequence.

In step 5, if any branches are present, these are preferably defined. In addition, the SU at the junction of each branch is also identified. In step 6, for such branched structures, all of the possible linear sequences which could be obtained from the branched CC are preferably determined, along with the SU at the junction of each branch. More preferably, each such sequence then receives an identification number. Preferably, the user is then able to select which such sequences should be used for the process of comparison.

In step 7, the query sequence “slides” along the target sequence, such that each SU of the query sequence is compared to the SU at the corresponding position of the target sequence. This step preferably involves the steps of a method for comparing the saccharide units as follows. The basic SU comparison is made by comparing each of the three elements which constitute the SU:

1. The sugar and its modifications (if any)

2. The anomerity of the glycosidic bond that connects the sugar to its neighbor

3. The position by which the sugar is connected to its neighbor

The score for each element comparison is determined through the use of comparison tables that take into account the stereo-chemical structure of the sugar and its modification. The final SU similarity score is the weighted average of the three elements.

As an example, the monosaccharides are preferably compared as follows. First the structure type of the monosaccharides are compared; if it is different the result of this comparison is a zero score. If the structure type is the same, the appropriate modifications of each monsaccharide are located in the appropriate entries of the table (according to the modification position). Then each entry is compared to its parallel entry for the other sequence of the pair, and a result is given according to the modification comparison table and the modification orientation comparison table.

Optionally and more preferably, the SU comparison can be made using a more complicated set of conditions that take into account additional considerations relevant to the compared SU, such as distance from branching position, synthesis by the same enzyme, and so forth. Most preferably, this more advanced comparison is performed according to the request of the user. An example of such an advanced comparison is described in greater detail with regard to Section 3 below.

In step 8, a score is determined for each particular orientation (sliding value) of the query sequence against the target sequence.

The three saccharide units which compose the branch junction are also optionally analyzed. The similarity between each junction in the query and target complex carbohydrate sequences is then preferably checked. This comparison is preferably performed as described in greater detail with regard to Section 4.

In step 9, the particular orientation of the sequences is preferably further analyzed to find the elements of sequence similarity, as well as the order. In order to determine this score, the results of each “sliding value” are preferably further processed in order to identify sub-sequence elements of similarity. The similarity element is a cluster of saccharide units with a similarity score above the threshold.

The similarity score threshold is preferably determined from the contents of the database. For example, optionally every saccharide unit in the database could be compared against every other saccharide unit, and an average value for the similarity scores is then determined. The similarity score threshold is then preferably set to be higher than this average value. Since the value for this threshold is also partially determined by the parameters which are set by the user, more preferably this value is recalculated after changing one or more of these parameters.

In step 10, the orientation of the query sequence is moved by one SU along the target sequence, and steps 7-9 are preferably repeated. More preferably, steps 7-10 are repeated until each possible orientation of the query sequence with regard to the target sequence is examined. However, preferably if the target and/or query sequence features at least one branch, the resultant plurality of linear sequences, which is derived from the branched sequence, is compared such that the common portions of the linear sequences are only compared once, to avoid weighting the score toward these portions.

In step 11, optionally and preferably, the similarity element is preferably further processed, in order to identify whether the relative position in the query sequence and the target sequence is the same.

In step 12, the final homology score is preferably calculated as the sum of the similarity element scores. Each element is given a bonus for correct length and order; the score is reduced for similar elements that are shifted. In addition, optionally the distance of a pair of similarity elements from the non-conjugated, or “free”, end of the complex carbohydrate is also considered as a factor. Preferably, a lower distance receives a higher score, since from a biological perspective, the non-conjugated end of the complex carbohydrate may be more available for interactions with proteins, such as carbohydrate receptors for example.

The score for junction similarity is added, and the final score is normalized according to the length of the CC.

The homology comparison pair may optionally be displayed with the query and the target CC above each other. The elements of similarity are preferably highlighted according to degree of similarity. Similarity elements in the target CC are preferably shifted and spaced so as to be under the matching elements from the query CC in the display.

Section 3: Comparison Scores for Saccharide Unit Pair

The score for the basic comparison of the saccharide units is preferably calculated as the sum of the comparisons scores of the three elements of the saccharide units as follows: the similarity score of the two monosaccharides and their modifications; the similarity score for the position by which the saccharide unit is connected to the neighboring saccharide unit; and the similarity score for the anomerity of the glycosidic bond that connects the saccharide unit to the neighboring saccharide unit.

According to preferred embodiments of the present invention, the factors and parameters that define the weight of each part could optionally be changed, either by the administrators, or by the user, in order to allow control on the sensitivity and fine tuning of the searching tools, for specific queries. The various factors and parameters are then optionally located in a definitions file.

More specifically, preferably the monosaccharides are compared according to their characters as described in the Monosaccharide (MS) Description Table. First, the structure type of the monosaccharides is compared; if the structure type is different, the result of the comparison is preferably a score of zero. Next, if the structure type is the same, the appropriate modifications of each monosaccharide are optionally located in the appropriate entries of the table (according to the modification position). Then each entry is compared to the parallel entry for the saccharide unit of the other sequence, and a result is preferably given according to the modification comparison table and the modification orientation comparison table.

For example, the comparison between Galactose (A) and N-acetyl Glucosamine (GN or G[2N] in full) is performed as follows.

The Galactose description according to the monosaccharide table is:

The Glucose description according to the MS table is:

| | Linear Code | Structure Type | 1 | 2 | 3 | 4 | 5 | 6 |
|---|---|---|---|---|---|---|---|---|
| D-Glucose | G | Dp | | EqD | EqU | EqD | EqU | |
| Modification | | | | OH | OH | OH | Y | OH |
| D-Galactose | A | Dp | | EqD | EqU | AxU | EqU | |
| !! ! ! ! ! ! ! ! !! | | | | OH | OH | OH | Y | OH |
| ! | | | | | | | | |

For GN or G[2N] the modification in position number 2 is changed from OH to N:

| | Linear Code | Structure Type | 1 | 2 | 3 | 4 | 5 | 6 |
|---|---|---|---|---|---|---|---|---|
| D-Glucose | G[2N] | Dp | | EqD | EqU | EqD | EqU | |
| Modification | | | | N | OH | OH | Y | OH |

The two monosaccharides (A and G) have the same structural type (Dp); therefore the comparison is meaningful. For each position the modification and the modification orientation are compared, and a value is given according to the modification comparison table and the modification orientation comparison table. The results are multiplied and summed.

| | | | | | | | | | |
|---|---|---|---|---|---|---|---|---|---|
| Position | 1 | 2 | 3 | 4 | 5 | 6 | 7 | 8 | 9 |

| | | | | | |
|---|---|---|---|---|---|
| A) Modification orientation comparison | 1 | 1 | 0 | 1 | 1 |
| score | | | | | |
| B) Modification comparison score | 0 | 1 | 1 | 1 | 1 |
| Final = B * A | 0 | 1 | 0 | 1 | 1 |

The final score is (1 + 1 + 1)/5 = 3/5

Next, the anomers are preferably compared. If the anomers are the same the score is preferably set to 1; if they are dissimilar, the score is preferably set to 0.

Next, the position of the connection between the monosaccharide and the neighboring saccharide unit is compared. If the connection is at the same position, the score is preferably set to 1; if they are dissimilar, the score is preferably set to 0.

The final comparison score is preferably calculated by multiplying each of the above scores (monosaccharide and modifications, anomer and position) with a factor that is preferably defined by the user, which acts as a weight for determining the importance given to each element. A final score between 0-1 is then accepted.

The table below of a comparison between of some common monosaccharides is given as an example only of such comparisons, and is not intended to be limiting in any way.

Monosaccharide Description Table

| Monosaccharide | Linearcode | | 1 | 2 | 3 | 4 | 5 | 6 | 7 | 8 | 9 |
|---|---|---|---|---|---|---|---|---|---|---|---|
| D-Glucose | G | Dp | | EqD | EqU | EqD | EqU | | | | |
| Modification Type | | | | OH | OH | OH | Y | OH | | | |
| L-Fucose | F | Lp | | EqU | EqD | AxD | EqD | | | | |
| Modification Type | | | | OH | OH | OH | Y | M | | | |
| D-Galactosce | A | Dp | | EqD | EqU | AxU | EqU | | | | |
| Modification Type | | | | OH | OH | OH | Y | OH | | | |
| D-Mannose | M | Dp | | AxU | EqU | EqD | EqU | | | | |
| Modification Type | | | | OH | OH | OH | Y | OH | | | |
| D-Xylose | X | Dp | | EqD | EqU | EqD | | | | | |
| Modification Type | | | | OH | OH | OH | Y | | | | |
| D-Arabinose | R | Dp | | EqU | EqD | AxD | | | | | |
| Modification Type | | | | OH | OH | OH | Y | | | | |
| L-Rhamnose | H | Lp | | AxD | EqD | EqU | EqD | | | | |
| Modification Type | | | | OH | OH | OH | Y | M | | | |
| D-Rhamnose | H* | Dp | | AxU | EqU | EqD | EqU | | | | |
| Modification Type | | | | OH | OH | OH | Y | M | | | |
| D-Glucuronic | U | Dp | | EqD | EqU | EqD | EqU | | | | |
| Modification Type | | | | OH | OH | OH | Y | OOH | | | |
| L-Iduronic acid | I | Lp | | EqU | EqD | EqU | EqD | | | | |
| Modification Type | | | | OH | OH | OH | Y | OOH | | | |
| Neuraminic acid | N | Lp | EqD | | EqD | EqU | EqD | | | | |
| Modification Type | | | OOH | Y | OH | N | Y | OH | OH | OH | |
| D-Galacuronic acid | L | Dp | | EqD | EqU | AxU | EqU | | | | |
| Modification Type | | | | OH | OH | OH | Y | OH | | | |
| KDN | K | | EqU | | EqD | EqU | EuD | | | | |
| Modification Type | | | OOH | Y | OH | OH | Y | OH | OH | OH | |
| D-Ribofuranose | B | Df | | AxD | AxD | AxU | | | | | |
| Modification Type | | | | OH | OH | Y | OH | | | | |

Dp=D pyranose, Df=D furanose, Lp=L pyranose, Lf=L furanose, EqD=Equatorially down, EqU=Equatorially up, AxD=Axially down, AxU=Axially up

The next table, for the comparison of the orientation of sugar modifications, is again given as an example only, without any intention of being limiting.

Modification Orientation Comparison Table

| | AxU | AxD | EqU | EqD | nothing |
|---|---|---|---|---|---|
| AxU | 1 | | | | |
| AxD | 0 | 1 | | | |
| EqU | 0.2 | 0 | 1 | | |
| EqD | 0 | 0.2 | 0.2 | 1 | |
| nothing | 0 | 0 | 0 | 0 | 1 |

The final table, for the comparison of the actual sugar modifications, is again given as an example only, without any intention of being limiting.

Saccharide Modifications and Comparison Table

| | Y | OH | O | V | S | SH | P | PN | PO | CH | Q | J | LL | N | T | E | CHO | C | D | OOH | nothing |
|---|---|---|---|---|---|---|---|---|---|---|---|---|---|---|---|---|---|---|---|---|---|
| Y | 1 | | | | | | | | | | | | | | | | | | | | |
| OH | 0 | 1 | | | | | | | | | | | | | | | | | | | |
| O | 0 | 0.8 | 1 | | | | | | | | | | | | | | | | | | |
| V | 0 | 0.2 | 0.2 | 1 | | | | | | | | | | | | | | | | | |
| S | 0 | 0.2 | 0.2 | 0.8 | 1 | | | | | | | | | | | | | | | | |
| SH | 0 | 0.2 | 0.2 | 0.2 | 0.2 | 1 | | | | | | | | | | | | | | | |
| P | 0 | 0.2 | 0.2 | 0.8 | 0.8 | 0 | 1 | | | | | | | | | | | | | | |
| PN | 0 | 0.2 | 0.2 | 0.2 | 0 | 0 | 0.2 | 1 | | | | | | | | | | | | | |
| PO | 0 | 0.2 | 0.2 | 0.2 | 0 | 0 | 0.2 | 0.8 | 1 | | | | | | | | | | | | |
| CH | 0 | 0 | 0 | 0 | 0 | 0 | 0 | 0 | | 1 | | | | | | | | | | | |
| Q | 0 | 0.2 | 0.2 | 0 | 0 | 0 | 0 | 0 | | | 1 | | | | | | | | | | |
| J | 0 | 0 | 0.2 | 0 | 0 | 0 | 0 | 0 | | | | 1 | | | | | | | | | |
| LL | 0 | 0 | 0.2 | 0 | 0 | 0 | 0 | 0 | | | | | 1 | | | | | | | | |
| N | 0 | 0 | 0.2 | 0 | 0 | 0 | 0 | 0 | | | | | | 1 | | | | | | | |
| T | 0 | 0 | 0.2 | 0 | 0 | 0 | 0 | 0 | | | | | | | 1 | | | | | | |
| E | 0 | 0 | 0 | 0 | 0 | 0 | 0 | 0 | | | | | | | | 1 | | | | | |
| CHO | 0 | 0 | 0 | 0 | 0 | 0 | 0 | 0 | | | | | | | | 0 | 1 | | | | |
| C | 0 | 0 | 0 | 0 | 0 | 0 | 0 | 0 | | | | | | | | 0 | 0.6 | 1 | | | |
| D | 0 | 0 | 0 | 0 | 0 | 0. | 0 | 0 | | | | | | | | 0 | 0.6 | 0.6 | 1 | | |
| OOH | 0 | 0.2 | 0.2 | 0.8 | 0. | 0 | 0.08 | 0 | | | | | | | | 0 | 0 | 0 | 0 | 1 | |
| Nothing | 0 | 0 | 0 | 0 | 0 | 0 | 0 | 0 | 0 | 0 | 0 | 0 | 0 | 0 | 0 | 0 | 0 | 0 | 0 | 0 | 1 |

An example for comparing two sequences with such advanced information is as follows. The similarity score is calculated for the following position:

- - - Ab3 (Fa4) GNb3- - -

- - - Ab4 (Fa3) GNb3- - -

According to the basic comparison, and giving equal weight to each part of the SU, the similarities score is:

- - - Ab3 (Fa4) GNb3- - -

- - - Ab4 (Fa3) GNb3- - -

- - - 0.66 0.66 1 - - -

But since it is known that the Fucose alpha 3 and alpha 4 branching are synthesized by the same glycosyltransferase enzyme and the branching position is determined according to the free position, 3 or 4 on the GN (Brent, 1992), the fucose SU in this context should receive a higher similarity score that the 0.66. If the user choose to use the advanced comparison this fucose SU receives a higher similarity score.

Section 4: Comparison of Junctions

The process of comparing junctions is preferably performed as follows. The first saccharide unit from one junction is compared to the first saccharide unit from the other junction, using the regular saccharide unit comparison method, as previously described. Next, the other saccharide units which form the junction are compared. Each junction has two possible forms for the comparison, such that the form which receives the highest score is preferably added to the score of the first SU. For example, for the comparison of the junctions—

Fa4 (ANb3) ANb4

ANa3 (Fa2) ANa3

The following pairs of the saccharide units are preferably compared:

| | | | | |
|---|---|---|---|---|
| ANb4 | [symbol] | ANa3 = | 0.33 | (the first SU) |
| ANb3 | [symbol] | Fa2 = | 0 | (the first form) |
| Fa4 | [symbol] | ANa3 = | 0 | (the first form) |
| ANb3 | [symbol] | ANa3 = | 0.66 | (the second form) |
| Fa4 | [symbol] | Fa2 = | 0.66 | (the second form) |

The first form=0

The second form=1.212

So the second form is preferably added to the first SU, to create the junction's score, which is 1.212 in this example (the maximum is 3 according to the exemplary score which is used herein).

The score is also preferably examined to determine if it is greater than the homology threshold, since only junctions with scores over this threshold are defined as similar junctions and added to the final calculation, in order to increase the homology score. In this example the value for the junctions is 3*0.4=1.2 so it is added to the final calculation.

Next, if the score is greater than the threshold, then the score is more preferably factored by the percentage from the total junctions. For example, if one sequence has 2 junctions, in which one of them is identical to the sole junction of the other sequence, the score will be multiplied by ½, whereas if both the sequences have only one junction, the score for the junction is not changed.

If similar junctions are in the same distance from the free or conjugated end of the target and query sequences, their score is increased by a factor that is controlled by the user.

The score is optionally factored by the length of the sequence, with a factor which is preferably proportional to the length of the sequence.

Section 5: Further Analysis of Similarity Elements

In order to emphasize longer sequences of similar monosaccharides (similarity elements), clusters of similar saccharides are preferably identified. The process of identification is optionally and preferably performed as follows.

First, for each row in the individual slide results table, when a saccharide unit comparison score above the threshold level is identified, a similarity element is defined. The number of elements is then counted until a comparison score which is below the threshold level is identified. The count for the particular similarity element is thus determined.

For example, if a row in the slide result table has the following values: 0, 0.8, 1, 1, 0.2, 1, 0.2, 0.8, 0.7, and the threshold value is 0.3, then three similarity elements can be defined (shown with underlined text): 0, 0.8, 1, 1, 0.2, 1, 0.2, 0.8, 0.7.

Next, the order of the similarity elements is preferably determined. In particular, if there are 2 similarity elements in the sequences which are in reverse order, relative to each other, then the score is preferably reduced for this comparison, while if they are in the correct order, the score is preferably increased.

The following example demonstrates this process for these sequences:

Query Sequence Ab2 Aa2 Fa2 Ga2 Gb3 CCID=Y

Target Sequence Ga2 Gb3 Fa2 Ab2 Aa2 CCID=X

Sliding result table:

| | | Query | | | | | |
|---|---|---|---|---|---|---|---|
| | | SU No. | | | | | |
| Query | Target | Slide | | | | | |
| CC ID | CC ID | value | 5 | 4 | 3 | 2 | 1 |
| Y | X | −4 | | | | | 0.33 |
| Y | X | −3 | | | | 1 | 1 |
| Y | X | −2 | | | 0.66 | 0.33 | 0 |
| Y | X | −1 | | 0.86 | 0 | 0.66 | 0.6 |
| Y | X | 0 | 0.6 | 0.26 | 1 | 0.6 | 0.266 |
| Y | X | 1 | 0.6 | 0.66 | 0.33 | 0.866 | |
| Y | X | 2 | 0.33 | 0.66 | 0.66 | | |
| Y | X | 3 | 1 | 1 | | | |
| Y | X | 4 | 0.66 | | | | |

The similarity elements are located as previously described, with a threshold minimum value of 0.7. Next, each pair of elements with the underline marking is examined, if they have different slide values and start at different positions, as determined by the position of the first left saccharide unit of the similarity element, to see if they are reversed.

Such an examination is performed by comparing the relationship between the original positions, according to the serial number of the first left saccharide unit of the similarity elements, to determine if the relationship is maintained after subtracting the slide value from the position of each similarity element.

For example, if the original positions are 1<4, and after subtraction have become (1-−3)>(4−3), the sign has changed, indicating that the order of this pair is reversed.

Section 6: Specific Example of Analysis Method

This section describes the steps of a specific example for the method of analysis according to the present invention. In addition, this example describes the operation of the method of comparison, as well as the determination of homology scores, with presentation of the results, according to the present invention. A query complex carbohydrate sequence (CCID=X) and target complex carbohydrate sequence (CCID=Y). The CCID is the identifier for each complex carbohydrate sequence, as these sequences are available from a database in this example.

CCID=X Ab4(Fa3)GNb3Ab4 GNb3Ab4GNb3Ab4GbC

CCID=Y Ab4GNb3(Ab4GNb6)Ab4GNb3Ab4GbC

The first stage is to divide the complex carbohydrate to the component saccharide units, and to determine all of the component linear sequences if one or more branches are present. In addition, the abbreviation for each modification is preferably written out in the full version of the name.

Next, the component linear sequence for each branch of the overall sequence is then determined, and assigned a separate identifier. The parentheses remain marked in order to show the junction point for the branch, as shown below:

CCID=X.1 Ab4) G[2N]b3 Ab4 G[2N]b3 Ab4 G[2N]b3 Ab4 GbC

CCID=X.2 Fa3) G[2N]b3 Ab4 G[2N]b3 Ab4 G[2N]b3 Ab4 GbC

CCID=Y.1 Ab4 G[2N]b6 )Ab4 G[2N]b3 Ab4 GbC

CCID=Y.2 Ab4 G[2N]b3 )Ab4 G[2N]b3 Ab4 GbC

The next stage is to “slide” each sequence against the other, and to calculate the slide result table, which is the comparison result for each orientation of the query sequence to the target sequence. For this example, the position, anomerity and monosaccharide type with the modification have the same weight in the similarity score calculations. One exception to this comparison is that each portion of a branched sequence before the branch point is preferably only compared once, in order to avoid weighting the score toward these common portions.

The next stage is to identify the similarity elements according to the threshold value (TV=0.689) and mark them (marked by under line in the slide result table).

Slide results table:

| | | Query CC | | | | | | | | |
|---|---|---|---|---|---|---|---|---|---|---|
| Query | Target | SU No. | | | | | | | | |
| CC ID | CC ID | Slide value | 8 | 7 | 6 | 5 | 4 | 3 | 2 | 1 |
| X.1 | Y.1 | −5 | | | | | | | | 0.6 |
| X.1 | Y.1 | −4 | | | | | | | 1 | 0.6 |
| X.1 | Y.1 | −3 | | | | | | 0.53 | 0.53 | 0.6 |
| X.1 | Y.1 | −2 | | | | | 1 | 1 | 1 | 0.6 |
| X.1 | Y.1 | −1 | | | | 0.53 | 0.53 | 0.53 | 0.53 | 0.6 |
| X.1 | Y.1 | 0 | | | 1 | 1 | 1 | 1 | 1 | 1 |
| X.1 | Y.1 | 1 | | 0.53 | 0.53 | 0.53 | 0.53 | 0.53 | 0.6 | |
| X.1 | Y.1 | 2 | 1 | 1 | 1 | 1 | 1 | 0.53 | | |
| X.1 | Y.1 | 3 | 0.53 | 0.53 | 0.53 | 0.53 | 0.6 | | | |
| X.1 | Y.1 | 4 | 1 | 1 | 1 | 0.6 | | | | |
| X.1 | Y.1 | 5 | 0.53 | 0.53 | 0.6 | | | | | |
| X.1 | Y.1 | 6 | 1 | 0.6 | | | | | | |
| X.1 | Y.1 | 7 | 0.6 | | | | | | | |
| X.1 | Y.2 | −5 | | | | | | | | 0.6 |
| X.1 | Y.2 | −4 | | | | | | | 1 | 0.6 |
| X.1 | Y.2 | −3 | | | | | | 0.53 | 0.53 | 0.6 |
| X.1 | Y.2 | −2 | | | | | 1 | 0.66 | 1 | 0.6 |
| X.1 | Y.2 | −1 | | | | 0.53 | 0.53 | 0.53 | 0.53 | 0.6 |
| X.1 | Y.2 | 0 | | | 1 | 0.66 | 1 | 1 | 1 | 1 |
| X.1 | Y.2 | 1 | | 0.53 | 0.53 | 0.53 | 0.53 | 0.53 | 0.6 | |
| X.1 | Y.2 | 2 | 1 | 0.6 | 1 | 1 | 1 | 0.6 | | |
| X.1 | Y.2 | 3 | 0.53 | 0.53 | 0.53 | 0.53 | 0.6 | | | |
| X.2 | Y.1 | 2 | 0 | 1 | 1 | 1 | 1 | 0.6 | | |
| X.2 | Y.1 | 3 | 0.33 | 0.53 | 0.53 | 0.53 | 0.6 | | | |
| X.2 | Y.1 | 4 | 0 | 1 | 1 | 0.6 | | | | |
| X.2 | Y.1 | 5 | 0.33 | 0.53 | 0.6 | | | | | |
| X.2 | Y.1 | 6 | 0 | 0.6 | | | | | | |
| X.2 | Y.1 | 7 | 0.6 | | | | | | | |
| X.2 | Y.2 | 2 | 0 | 0.66 | 1 | 1 | 1 | 0.6 | | |
| X.2 | Y.2 | 3 | 0 | 0.53 | 0.53 | 0.53 | 0.6 | | | |

The next stage is to compare the junction elements:

CCID=X Junction 1 A b4 (Fa3) GNb3

CCID=Y Junction 1 GNb6 (GNb3)Ab4

The score for the second form is:

A b4 (F a3) G[2N]b3

G[2N]b3 (G[2N]b6) A b4

0.53+0+0.53=1.06

The score for the first form is:

F a3 (A b4) G[2N]b3

G[2N]b3 (G[2N]b6) A b4

0.33+0.53+0.53=1.54

The threshold for the junction is the sum of the minimum threshold value for each of three saccharide units, or 3*0.689=2.067. In both forms, the total score is therefore less than this threshold value. Thus, the score for the junctions is not included in the final score for this example.

For the final score calculation, each similarity element score is factored as follows. First, any decrease in the similarity element score according to the number of elements in the “slide”, or slide value (SV), is calculated according to a proportional factor. The SVDF (Slide Value Decreasing Factor) is preferably determined by the user, and lies in the range of 0 to 1. Therefore, the decreased value is calculated as −(SVDF)*(absolute value of SV).

Next, the score may optionally be increased according to the length of the similarity element as previously described. Preferably, the increase to the score is calculated as (LIF)*(length of similarity element)², in which LIF is the Length Increasing Factor, 0<LIF<1. The LIF is preferably determined by the user.

Then, optionally and more preferably, the similarity score is increased according to the distance of the similarity element from the non conjugated end of the complex carbohydrate. Preferably, the increase to the score is calculated as (DFEIF)/(1+distance from query complex carbohydrate end+distance from target complex carbohydrate end), in which DFEIF is the Distance From End Increasing Factor, DFEIF>1. The DFEIF is preferably determined by the user.

Finally, optionally and more preferably, the similarity score is also decreased according to the RODF (Reverse Order Decreasing Factor), which is greater than zero, and which is preferably determined by the user.

In this example, the following values of factors are used:

SVDF=0.2

LIF=0.1

DFEIF=2

RODF=1

All of the increasing and decreasing values are summed and added to the original similarity score.

As an example the calculation for following similarity element:

| | | SV | 8 | 7 | 6 | 5 | 4 | 3 | 2 | 1 |
|---|---|---|---|---|---|---|---|---|---|---|
| X.1 | Y.1 | 0 | | | 1 | 1 | 1 | 1 | 1 | 1 |

is preferably performed as shown in the table below.

| | | | | Decreasing | |
|---|---|---|---|---|---|
| | Decreasing | Increasing | Increasing due | Due to | |
| Original | Due to | due to | to distance | Reverse | |
| score | Slide Value | Length | from the end | order | Final score |
| 6+ | (−0.2 * 0) + | (0.1 * 6²) + | (2/(1 + 2 + 2) + | 0= | 10 |

All of the factored similarity element scores are then preferably summed and divided by the length of the target complex carbohydrate, as measured in saccharide units, to give the final similarity score.

The process is preferably repeated for all the target complex carbohydrates, and the results are shown to the user. The target complex carbohydrates with the highest score are the most similar to the query complex carbohydrate. The complex carbohydrate similarity score is meaningful only when compared to other similarity scores which are calculated in the same way.

Next, optionally and preferably, the best fit alignments for the CCID=X and CCID=Y is calculated by creating all of the possible combinations of shifting and spacing of the target sequence and query sequence, and summing the similarity score for every fit. The results are preferably displayed in decreasing order and the similarity elements with the highest scores are optionally marked with different colors, or, as in the example below, with underlining or other font formatting.

Ab4(Fa3)GNb3Ab4 GNb3Ab4GNb3Ab4GbC

Ab4 GNb3(Ab4GNb6)Ab4GNb3Ab4GbC

Ab4(Fa3)GNb3Ab4GNb3Ab4GNb3Ab4GbC

Ab4 GNb3(Ab4 GNb6)Ab4GNb3Ab4GbC

Section 7: Exemplary System for Sequence Analysis

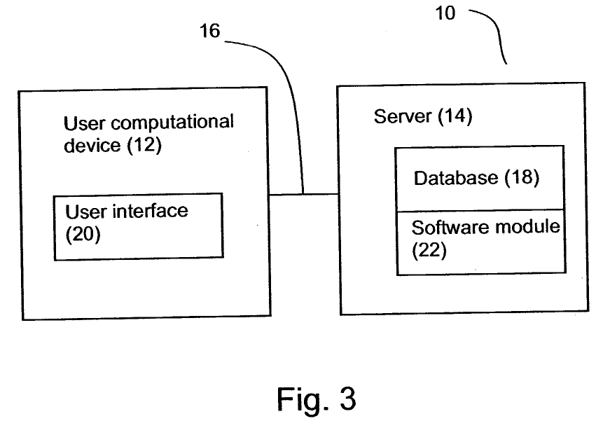

FIG. 3 is a schematic block diagram of an exemplary system according to the present invention for carbohydrate sequence analysis. As shown, a system 10 includes a user computational device 12 for operation by a user (not shown), which is connected to a server 14 through a network 16. Network 16 could be the Internet, for example. Server 14 controls the operation of a database 18, which contains a plurality of complex carbohydrate sequences in the format of the present invention.

The operation of system 10 is as follows. When the user wishes to perform a search for a query complex carbohydrate sequence, the user preferably enters the sequence through a user interface 20, which is provided through user computational device 12. The query sequence, optionally with any user-defined parameters, is then sent to server 14 through network 16. The method of the present invention for comparing carbohydrate sequences is then preferably performed as previously described, for example by a software module 22 being operated by server 14. The results of the search and comparison are then sent to user computational device 12, and are preferably displayed through user interface 20.

### REFERENCES

Abbott A (1999) Nature 398:6729 646

Bohne, A., Lang, E. and von der Lieth, C. W. (1998) J. Mol. Model. 4 33-43.

Brazma A, Jonassen I, Eidhammer I, Gilbert D (1998) J Comput Biol Summer 5 279-305.

Bruno, I. J., Kemp, N. M., Artymiuk, P. J. and Willett, P. (1997) Carbohydrate res. 304, 61-67.

Bush C A, Martin-Pastor M, Imberty A (1999) Annu Rev Biophys Biomol Struct 28 269-93.

B W Weston, R P Nair, R D Larsen, and J B Lowe, (1992) JBC 267 4152-4160.

Davis, B. G. (2000), Chem & Industry 21 134-139

Dwek, R. A., (1996) Chem. Rev., 96 683-720.

Frishman D, Heumann K, Lesk A, Mewes H W (1998) Bioinformatics 14 551-61

Gershon D, Sobral B W, Horton B, Wickware P, Gavaghan H, Strobl M (1997) Nature 389:6649 417-22

Gohier A, Espinosa J F, Jimenez-Barbero J, Carrupt P A, Prez S, Imberty A (1996) J Mol Graph 14 322-7, 363-4.

Gotoh O, (1999) Adv Biophys 36 159-206

Imberty A, Monier C, Bettler E, Morera S, Freemont P, Sippl M, Flyckner H, R□ger W, Breton C (1999) Glycobiology9 713-22.

Knapman, K. Informatics, In: www.chemweb.com/alchemy/1999/molmodel, June 11,.

Koch A E, Halloran M M, Haskell C J, Shah M R, Polverini P J, (1995)Nature 376:6540 517-9.

Laine, R. A. (1994) Glycobiology 4 759-767.

Ouellette F (1999) Clin Genet 56 179-85

Persidis A (1999) Nat Biotechnol 17 828-30

R Sawada, S Tsuboi, and M Fukuda (1994) JBC 269 1425-1431.

Rawlings C J, Searls D B (1997) Curr Opin Genet Dev 7416-23

Searls, D. B. (2000) Drug Discovery Today 5 135-143.

Sharon, N. (1975) In: Complex Carbohydrates: Their Chemistry, Biosynthesis and Functions. Eds: Addison-Wesley Publishing Company, USA.

Thayer, A. M. (2000), C&EN 78 19-32.

Ullmann, J. R. (1976) J. Association Computing Machinery 23 31-42.

von der Lieth C, Siebert H, Kozar T, Burchert M, Frank M, Gilleron M, Kaltner H, Kayser G, Tajkhorshid E, Bovin N V, Vliegenthart J F, Gabius H (1998) Acta Anat (Basel) 16191-109.

von Itzstein M, Wu W Y, Kok G B, Pegg M S, Dyason J C, Jin B, Van Phan T, Smythe M L, White H F, Oliver S W, (1993) Nature 363:6428 418-23.

What is claimed is:

1. A method of representing a carbohydrate structure as a linear sequence of characters, the method being performed by a data processor, the method comprising:
(a)representing saccharide units of the carbohydrate structure with character codes, each character code defining:
(i)an identity of a saccharide unit; and
(ii)a presence or absence of a chemical modification to said saccharide unit and optionally a type of said chemical modification; and
(b)assembling said character codes into a linear string of characters being uniquely representative of the carbohydrate structure.

2. The method of claim 1, wherein step (b) includes adding to said linear string of characters additional characters defining a type of connection between adjacent saccharide units.

3. The method of claim 1, wherein step (b) includes adding to said linear string of characters additional characters defining at least one branch of the carbohydrate structure.

4. The method of claim 3, wherein said additional characters define a structure and/or a hierarchy of said at least one branch.

5. The method of claim 2, wherein said linear string of characters includes letters defining said saccharide units and syntax defining said type of connection between adjacent saccharide units.

6. The method of claim 3, wherein said at least one branch is defined by a start point character and an end point character.

7. The method of claim 1, wherein said character code is a one letter code.

8. The method of claim 1, wherein step (b) includes adding to said linear string of characters additional characters identifying uncertainties within the carbohydrate structure.

9. A method of representing a branched carbohydrate structure as a linear sequence of characters, the method being performed by a data processor, the method comprising:
(a)representing saccharide units of the branched carbohydrate structure with character codes, each of said character codes defining an identity of a specific saccharide unit;
(b)defining at least one branch of the branched carbohydrate with at least one branch specific character; and
(c)assembling said character codes and said at least one branch specific character into a linear string of characters being uniquely representative of the branched carbohydrate structure.

10. The method of claim 9, wherein step (b) is effected by defining a branch start point with a first branch specific character and branch end point with a second branch specific character.

11. The method of claim 9, wherein said at least one branch specific character defines a structure and/or a hierarchy of said at least one branch.

12. The method of claim 9, further comprising the step of defining a type of connection between said saccharide units of the branched carbohydrate structure prior to step (c).

13. The method of claim 12, wherein said linear string of characters includes letters defining said saccharide units and syntax defining said type of said connection between said saccharide units.

14. The method of claim 9, further comprising the step of adding to said linear string of characters additional characters identifying uncertainties within the branched carbohydrate structure.

15. A method of representing a partially characterized carbohydrate structure as a linear sequence of characters, the method being performed by a data processor, the method comprising:
(a)representing saccharide units of the carbohydrate structure with first character codes, each of said first character codes defining an identity of a specific saccharide unit;
(b)representing uncertainties within the carbohydrate structure with second character codes, each of said second character codes identifying a specific structural uncertainty; and
(c)assembling said first character codes and said second character codes into a linear string of characters being uniquely representative of the partially characterized carbohydrate structure.

16. The method of claim 15, further comprising the step of adding to said linear string of characters additional characters defining a type of connection between adjacent saccharide units.

17. The method of claim 15, further comprising the step of adding to said linear string of characters additional characters defining at least one branch of the carbohydrate structure.

18. The method of claim 17, wherein said additional characters define a structure and/or a hierarchy of said at least one branch.

19. The method of claim 16, wherein said linear string of characters includes letters defining said saccharide units and syntax defining said type of connection between adjacent saccharide units.

20. The method of claim 17, wherein said at least one branch is defined by a start point character and an end point character.

21. The method of claim 15, wherein said first and/or second character code is a one letter code.

22. A system for representing a carbohydrate structure as a linear sequence of characters, the system comprising:
(a)a representor designed for representing saccharide units of the carbohydrate structure with character codes, each character code defining:
(i)an identity of a saccharide unit; and
(ii)a presence or absence of a chemical modification to said saccharide unit and optionally a type of said chemical modification; and
(b)an assembler for assembling said character codes into a linear string of characters being uniquely representative of the carbohydrate structure.

23. The system of claim 22, wherein said representor is further designed for adding to said linear string of characters additional characters defining a type of connection between adjacent saccharide units.

24. The system of claim 22, wherein said representor is further designed for adding to said linear string of characters additional characters defining at least one branch of the carbohydrate structure.

25. The system of claim 24, wherein said additional characters define a structure and/or a hierarchy of said at least one branch.

26. The system of claim 23, wherein said representor utilizes letters to define said saccharide units and syntax to define said type of connection between adjacent saccharide units.

27. The system of claim 24, wherein said representor defines said at least one branch with a start point character and an end point character.

28. The system of claim 22, wherein said representor is further designed for adding to said linear string of characters additional characters identifying uncertainties within the carbohydrate structure.

29. A system for representing a branched carbohydrate structure as a linear sequence of characters, the system comprising:
(a) a representor designed for:
(i) representing saccharide units of the branched carbohydrate structure with character codes, each of said character codes defining an identity of a specific saccharide unit; and
(ii) defining at least one branch of the branched carbohydrate with at least one branch specific character; and
(b) an assembler designed for assembling said character codes and said at least one branch specific character to generate a linear string of characters being uniquely representative of the branched carbohydrate structure.

30. The system of claim 29, wherein said representor is designed for defining a branch start point with a first branch specific character and branch end point with a second branch specific character.

31. The system of claim 30, wherein said at least one branch specific character defines a structure and/or a hierarchy of said at least one branch.

32. The system of claim 29, wherein said representor is further designed for defining a type of connection between said saccharide units of the branched carbohydrate structure.

33. The system of claim 32, wherein said representor is designed for defining said saccharide units with letters and defining said type of said connection between said saccharide units with syntax.

34. The system of claim 29, wherein said representor is further designed for adding to said linear string of characters additional characters identifying uncertainties within the branched carbohydrate structure.

35. A system for representing a partially characterized carbohydrate structure as a linear sequence of characters, the system comprising:
(a) a representor designed for:
(i) representing saccharide units of the carbohydrate structure with first character codes, each of said first character codes defining an identity of a specific saccharide unit;
(ii) representing uncertainties within the carbohydrate structure with second character codes, each of said second character codes identifying a specific structural uncertainty; and
(b) an assembler designed for assembling said first character codes and said second character codes into a linear string of characters being uniquely representative of the partially characterized carbohydrate structure.

36. The method of claim 35, wherein said representor is further designed for adding to said linear string of characters additional characters defining a type of connection between adjacent saccharide units.

37. The method of claim 35, wherein said representor is further designed for adding to said linear string of characters additional characters defining at least one branch of the carbohydrate structure.

38. The system of claim 37, wherein said additional characters define a structure and/or a hierarchy of said at least one branch.

39. The method of claim 36, wherein said representor is designed for defining said saccharide units with letters and defining said type of said connection between said saccharide units with syntax.

40. The method of claim 37, wherein said at least one branch is defined by said representor using a start point character and an end point character.

41. The method of claim 35, wherein said first and/or second character code is a one letter code.
